(19) United States
(12) Patent Application Publication
(10) Pub. No.: US 2024/0362833 A1
(43) Pub. Date: Oct. 31, 2024

(54) PROVIDING A 3D RESULTS DATA SET

(71) Applicant: Siemens Healthineers AG, Forchheim (DE)

(72) Inventors: Oliver Hornung, Unterleinleiter (DE); Michael Manhart, Furth (DE); Markus Kowarschik, Erlangen (DE); Manuela Meier, Nurnberg (DE)

(21) Appl. No.: 18/635,226

(22) Filed: Apr. 15, 2024

(30) Foreign Application Priority Data
Apr. 28, 2023 (EP) 23170624.3

(51) Int. Cl. G06T 11/00; G06T 7/00

(52) U.S. Cl. CPC G06T 11/003; G06T 7/0012; G06T 2207/10116; G06T 2207/20084

(57) ABSTRACT

A computer-implemented method for providing a three-dimensional (3D) results data set includes: acquiring projection maps of an object under examination which are captured from various projection directions by a medical X-ray device; providing an initial projection matrix based on a static model of the X-ray device; providing a further projection matrix by applying a trained function to input data, wherein the input data is based on the initial projection matrix and the projection maps, wherein at least one parameter of the trained function is adapted based on an image quality metric and/or a consistency metric, and wherein the further projection matrix is provided as output data of the trained function; and providing the 3D results data set through reconstruction from the projection maps by the further projection matrix.

The present patent document claims the benefit of European Patent Application No. 23170624.3, filed Apr. 28, 2023, which is hereby incorporated by reference in its entirety.

### TECHNICAL FIELD

The present disclosure relates to a computer-implemented method for providing a three-dimensional (3D) results data set, a computer-implemented method for providing a trained function, a provision unit, a medical X-ray device, a training unit, and a computer program product.

### BACKGROUND

In cone-beam computed tomography (CBCT) for three-dimensional (3D) reconstruction of an object under examination from multiple projection directions, an “offline” calibration may firstly be carried out using a phantom of known geometry. With the assistance of two-dimensional (2D) projection maps of the phantom, 2D/3D correspondences may be determined and used to estimate projection matrices for each individual projection direction. The projection matrices may be stored in a database, for example. In the event of subsequent capture of projection maps of an unknown rigid object under examination from the same projection directions, the projection matrices may then be read out of the database for 3D reconstruction.

Use of the previously determined projection matrices for unknown objects under examination disadvantageously requires high reproducibility of an image chain and of movements of the imaging apparatus. This is associated with stringent requirements regarding the development and mechanical design of technical system components of the imaging apparatus. If system components of the imaging apparatus, (e.g., an X-ray tube, a drive, a transmission, and/or an X-ray detector), are changed, calibration has to be repeated from scratch. Calibration is time-consuming and cost-intensive and is moreover necessary for all projection directions of 3D capture protocols. 3D capture of projection maps is also disadvantageously bound to a calibrated isocenter position and the calibrated capture trajectory, in particular the projection directions.

### SUMMARY AND DESCRIPTION

It is therefore the object of the present disclosure to enable more flexible capture of projection maps of an object under examination and improved 3D reconstruction from the projection maps.

The scope of the present disclosure is defined solely by the appended claims and is not affected to any degree by the statements within this summary. The present embodiments may obviate one or more of the drawbacks or limitations in the related art.

This object is achieved as described below in relation both to methods and devices for providing a 3D results data set and to methods and devices for providing a trained function. Features, advantages, and alternative embodiments of data structures and/or functions in methods and devices for providing a 3D results data set may here be applied to analogous data structures and/or functions in methods and devices for providing a trained function. Analogous data structures may here in particular be characterized by the use of the qualifier “training.” Furthermore, the trained functions used in methods and devices for procedural support may have been adapted and/or provided by methods and systems for providing a trained function.

A first aspect of the disclosure relates to a computer-implemented method for providing a 3D results data set. In a first act, projection maps of an object under examination are acquired, which are captured from various projection directions, in particular along a 3D trajectory, by a medical X-ray device. Furthermore, an initial projection matrix is provided based on a static model of the X-ray device. A further projection matrix is additionally provided by applying a trained function to input data. In this case, the input data of the trained function is based on the initial projection matrix and the projection maps. Furthermore, at least one parameter of the trained function is adapted based on an image quality metric and/or a consistency metric. The further projection matrix is provided as output data of the trained function. Hereafter, the 3D results data set is provided through reconstruction from the projection maps by the further projection matrix.

The above-described acts of the proposed method may be partially or completely computer-implemented. In addition, the above-described acts of the proposed method are carried out at least partly, (e.g., wholly), in succession or at least partly simultaneously.

Acquisition of the projection maps may include receiving and/or capturing the projection maps. Receiving the projection maps may include acquiring and/or reading out a computer-readable data memory and/or receiving from a data storage unit, (e.g., a database), for example, by an interface.

The object under examination may be a human and/or animal patient and/or an examination phantom, in particular a vascular phantom.

The projection maps are captured from various projection directions along a 3D trajectory by the medical X-ray device, in particular a medical C-arm X-ray device. The various projection directions may advantageously be at least partly non-collinear. Furthermore, the projection directions may each describe a course of a beam, (e.g., a central and/or mid-beam), between an X-ray source and an X-ray detector, (e.g., a detector midpoint), of the X-ray device at the capture time of the respective projection maps.

In particular, the projection directions may in each case describe angulation of the X-ray device relative to the object under examination and/or an isocenter, in particular a center of rotation, of a defined arrangement of X-ray source and X-ray detector. The isocenter may describe a spatial point about which the defined arrangement of X-ray source and X-ray detector may be moved, (e.g., rotated), in particular during capture of the projection maps. Advantageously, the various projection directions may in each case extend through the isocenter (e.g., common isocenter). The 3D trajectory may describe a spatial path for arrangement of a reference point, e.g., a focal point, of the defined arrangement of X-ray source and X-ray detector. Advantageously, the multiple projection maps may in each case take the form of 2D spatially resolved maps of the object under examination.

The at least one initial projection matrix, in particular multiple initial projection matrices, are provided based on a static model of the X-ray device. The static model may include a physical and/or mathematical representation of static effects of the X-ray device, e.g., a digital twin and/or computer-aided model (computer-aided design, CAD). The static model may be determined using geometric features of the X-ray device and the components thereof. The static model may advantageously be received. Receiving the static model may include acquiring and/or reading out a computer-readable data memory and/or receiving from a data storage unit, (e.g., a database), such as by an interface. The static model of the X-ray device may be used to determine a mapping geometry between the X-ray source and the X-ray detector. The initial projection matrix may advantageously be determined using the mapping geometry, in particular as a function of the particular projection direction. In this case, the initial projection matrix may include a mapping rule for mapping 3D object points of the object under examination onto 2D pixels of the projection maps.

By applying the trained function to the input data, the further projection matrix is provided. The trained function may advantageously be trained by a machine learning method. The trained function may be an in particular artificial neural network, in particular a convolutional neural network (CNN) or a network including a convolutional layer.

The trained function maps input data onto output data. The output data may furthermore be dependent on one or more parameters of the trained function. The one or more parameters of the trained function may be determined and/or adapted by training. Determination and/or adjustment of the one or more parameters of the trained function may be based on a pair of training input data and associated training output data, in particular comparison output data, wherein the trained function is applied to the training input data to generate training map data. In particular, determination and/or adjustment may be based on a comparison of the training map data and the training output data, in particular comparison output data. A trainable function, e.g., a function with one or more parameters which have not as yet been adjusted, may also be denoted a trained function. By adapting the one or more parameters of the trained function, in particular by training the trained function, the trained function may be trained to adapt to new circumstances and to identify and extrapolate patterns.

The trained function may be adapted using supervised learning, partially supervised learning, unsupervised learning, reinforcement learning, representation learning, and/or active learning. The at least one parameter of the trained function may be iteratively adapted using multiple training acts.

Other terms for trained functions are trained mapping rule, mapping rule with trained parameters, function with trained parameters, or machine learning algorithm. One example of a trained function is an artificial neural network, wherein edge weights of the artificial neural network correspond to the parameters of the trained function. The term “neural net” may also be used instead of the term “neural network.” In particular, a trained function may include a neural network, e.g., a deep artificial neural network, a convolutional neural network, a deep convolutional network, an adversarial network, a deep adversarial network, a generative adversarial network, a “Support Vector Machine,” a decision tree, and/or a Bayesian network. Alternatively or additionally, the trained function is based on k-means clustering, Q-learning, genetic algorithms, and/or association rules.

The trained function may be trained by way of backpropagation. First of all, training map data may be determined by applying the trained function to the training input data. A deviation between the training map data and the training output data, (e.g., the comparison output data), may then be ascertained by applying an error function to the training map data and the training output data, (e.g., the comparison output data). Furthermore, at least one parameter, (e.g., the weighting of the trained function), may be iteratively adapted. In this way, the deviation between the training map data and the training output data, (e.g., the comparison output data), may be minimized during training of the trained function.

The trained function, (e.g., the neural network), advantageously has an input layer and an output layer. The input layer may be configured to receive input data. The output layer may furthermore be configured to provide map data, in particular output data. The input layer and/or the output layer may in each case include a plurality of channels, in particular neurons.

The input data of the trained function is based on the procedural data. In particular, the input data of the trained function includes the procedural data. Furthermore, the trained function provides the time information and the target procedure configuration as output data. At least one parameter of the trained function is adapted based on a comparison of a training procedure configuration with a comparison procedure configuration and a comparison of training time information with comparison time information. In particular, the trained function may be provided by an embodiment of the proposed method for providing a trained function which is described further below.

The input data of the trained function is based on the initial projection matrix and the projection maps. In particular, the input data of the trained function includes the initial projection matrix and the projection maps. Furthermore, the trained function provides the further projection matrix as output data. At least one parameter of the trained function is adapted based on an image quality metric and/or a consistency metric. In particular, the trained function may be provided by an embodiment of the proposed method for providing a trained function which is described further below. The further projection matrix may include a mapping rule for mapping 3D object points of the object under examination onto 2D pixels of the projection maps. Advantageously, the 3D results data set is reconstructed from the plurality of projection maps by the further projection matrix, e.g., using a filtered backprojection.

The 3D results data set may map a plurality of pixels, (e.g., voxels), with image values, (e.g., intensity values and/or attenuation values), which map the object under examination in 3D spatially resolved manner.

Providing the 3D results data set may include storing on a computer-readable storage medium and/or display on a display unit and/or transfer to a provision unit. In particular, a graphical representation of the 3D results data set may be displayed by the display unit.

The proposed method may advantageously enable more flexible capture of the projection maps of the object under examination and improved 3D reconstruction of the 3D results data set from the projection maps.

In a further advantageous embodiment of the proposed method for providing a 3D results data set, one initial and one further projection matrix may be provided for each of the various projection directions. In this case, the input data of the trained function may be based on the initial projection matrices. Furthermore, the 3D results data set may be provided through reconstruction from the projection maps by the further projection matrices.

Advantageously, an initial projection matrix based on the static model of the X-ray device may be provided for each of the various projection directions of the plurality of projection directions. The input data of the trained function may advantageously be based on the plurality of initial projection matrices, in particular include the plurality of initial projection matrices. Furthermore, the trained function may provide a further projection matrix as output data for each of the various projection directions of the plurality of projection directions. Advantageously, the 3D results data set may be reconstructed from the plurality of projection maps, wherein the further projection matrix corresponding to the projection direction of the respective projection map is used.

The proposed embodiment may enable improved reconstruction of the 3D results data set, in particular a reconstruction configured to the various projection directions.

In a further advantageous embodiment of the proposed method for providing a 3D results data set, the input data of the trained function may additionally be based on the static model of the X-ray device.

Advantageously, the input data of the trained function may additionally include the static model of the X-ray device. In this way, the at least one further projection matrix may advantageously be provided while additionally taking account of the static model of the X-ray device.

In one further advantageous embodiment of the proposed method for providing a 3D results data set, information on dynamic degrees of freedom of movement of the X-ray device may be acquired which defines a latent space. The input data of the trained function may additionally be based on the latent space.

The medical X-ray device may have a plurality of dynamic, in particular nonlinear, degrees of freedom of movement. The dynamic degrees of freedom of movement may characterize an effect of friction and/or damping and/or vibration and/or moments of inertia and/or centrifugal effects and/or Coriolis effects on movement of the X-ray device. In particular, the dynamic degrees of freedom of movement may describe a maximum influence of friction and/or damping and/or vibration and/or moments of inertia and/or centrifugal effects and/or Coriolis effects on movement of the X-ray device. The latent space may be defined, (e.g., predetermined and/or delimited), by the dynamic degrees of freedom of movement, (e.g., also by static degrees of freedom of movement), of the X-ray device. The dynamic degrees of freedom of movement, (e.g., the latent space), may describe physically possible dynamic deviations from static movements of the X-ray device.

The dynamic degrees of freedom of movement, in particular the latent space, of the X-ray device may be learned, e.g., using a variational autoencoder. The dynamic degrees of freedom of movement, (e.g., the latent space), may advantageously be determined based on deviations between initial projection matrices, which are determined using the static model of the X-ray device, and calibration projection matrices, which are determined using calibration projection maps of a phantom of known geometry.

The input data of the trained function may advantageously additionally include the latent space, in particular information characterizing the latent space.

In this way, the at least one further projection matrix may advantageously be provided while additionally taking account of the dynamic degrees of freedom of movement of the X-ray device.

A second aspect relates to a computer-implemented method for providing a trained function. Training projection maps of a training object under examination that map the training object under examination are acquired from various projection directions, in particular along a 3D trajectory. In this case, the training projection maps are simulated or captured by a medical training X-ray device. In a further act, an initial training projection matrix is provided based on a static training model of the medical training X-ray device. Furthermore, a further training projection matrix is provided by applying the trained function to input data. In this case, the input data of the trained function is based on the training projection maps and the initial training projection matrix. The further training projection matrix is provided as output data of the trained function. In a further act, a 3D training data set is reconstructed from the training projection maps by the further training projection matrix. Furthermore, an evaluation parameter is determined in each case by applying an image quality metric and/or a consistency metric to the 3D training data set. At least one parameter of the trained function is then adapted based on a comparison of the evaluation parameter with a reference value. The trained function is furthermore provided.

The above-described acts of the proposed method may be partially or completely computer-implemented. In addition, the above-described acts of the proposed method are carried out at least partly, (e.g., wholly), in succession or at least partly simultaneously.

The training projection maps may advantageously have all the features and characteristics of the projection maps described in relation to the method for providing a 3D results data set and vice versa. Acquisition of the training projection maps may include receiving and/or capturing the projection maps. Receiving the training projection maps may include acquiring and/or reading out a computer-readable data memory and/or receiving from a data storage unit, (e.g., a database), such as by an interface.

According to a first variant, the training projection maps may be captured from the various projection directions, (e.g., along the 3D trajectory), by the medical training X-ray device, (e.g., a medical C-arm X-ray device). The various projection directions may advantageously be at least partly non-collinear.

The projection directions may each describe a course of a beam, (e.g., a central and/or mid-beam), between an X-ray source and an X-ray detector, (e.g., a detector midpoint), of the training X-ray device at the capture time of the respective training projection maps. In particular, the projection directions may describe angulation of the training X-ray device relative to the object under examination and/or an isocenter, (e.g., a center of rotation), of a defined arrangement of X-ray source and X-ray detector. The isocenter may describe a spatial point about which the defined arrangement of X-ray source and X-ray detector may be moved, (e.g., rotated), in particular during capture of the training projection maps. Advantageously, the various projection directions may in each case extend through the isocenter (e.g., common isocenter). The 3D trajectory may describe a spatial path for arrangement of a reference point, e.g., a focal point, of the defined arrangement of X-ray source and X-ray detector. Advantageously, the multiple training projection maps may take the form of 2D spatially resolved maps of the training object under examination.

Alternatively, the training projection maps may be simulated, e.g., by a virtual representation of the object under examination and a physical mapping model of the training X-ray device.

The training object under examination may advantageously have all the features and characteristics of the object under examination described in relation to the method for providing a 3D results data set and vice versa. The training object under examination may be the same as or different from the object under examination. In particular, the method for providing a trained function may be carried out repeatedly for different training objects under examination.

Provision of the initial training projection matrix based on the static training model of the training X-ray device may proceed in a similar way to provision of the initial projection matrix. The initial training projection matrix, the static training model, and the medical training X-ray device may in each case have all the features and characteristics of the initial projection matrix, the static model, and the medical X-ray device described in relation to the method for providing a 3D results data set and vice versa.

The further training projection matrix may be provided by applying the trained function to the input data. In this case, the input data of the trained function is based on the training projection maps and the initial projection matrix. In particular, the input data of the trained function may include the training projection maps and the initial projection matrix.

Advantageously, the 3D training data set may be reconstructed from the plurality of training projection maps by the further training projection matrix, e.g., by a filtered backprojection.

By applying the image quality metric and/or the consistency metric to the 3D training data set, it is advantageously possible to provide an evaluation parameter, in particular an image quality parameter and/or a consistency parameter. The evaluation parameter may evaluate, (e.g., qualitatively and/or quantitatively), the image quality and/or consistency of the 3D training data set.

Advantageously, the at least one parameter of the trained function may be adapted based on the comparison of the evaluation parameter with the predetermined reference value in such a way that, on repeated application of the trained function to the input data and reconstruction of the 3D training data set, the 3D training data set which may be provided has a higher image quality and/or consistency compared with the 3D training data set that was respectively previously provided. If a value of the evaluation parameter increases monotonically with increasing image quality and/or consistency, the at least one parameter of the trained function may advantageously be adapted in such a way that the evaluation parameter of the 3D training data set increases on repeated application of the trained function to the input data and reconstruction of the 3D training data set. If a value of the evaluation parameter falls monotonically with increasing image quality and/or consistency, the at least one parameter of the trained function may advantageously be adapted in such a way that the evaluation parameter of the 3D training data set falls on repeated application of the trained function to the input data and reconstruction of the 3D training data set.

The reference value may be received with the assistance of a user input acquired by an input unit. Alternatively or additionally, the reference value may be determined. The reference value may specify a threshold value for a minimum image quality and/or a minimum consistency of the 3D training data set. Advantageously, provision of the further training projection matrix, reconstruction of the 3D training data set, determination of the evaluation parameter, and adaptation of the at least one parameter of the trained function may be carried out repeatedly until the evaluation parameter reaches or exceeds the reference value.

Providing the trained function may include storage on a computer-readable storage medium and/or transfer to a provision unit. The proposed method may advantageously provide a trained function which may be used in an embodiment of the method for providing a 3D results data set.

The proposed method may advantageously provide a trained function that may be used in an embodiment of the method for providing a 3D results data set. Advantageously, performance criteria for the trained function may be independent of dedicated quality metrics applicable only for dedicated image content, instead being capable of being learned with the assistance of the training map pairs and left to the trained function.

In a further advantageous embodiment of the proposed method for providing a trained function, a 3D comparison data set may be reconstructed from the training projection maps by the initial training projection matrix. The reference value may moreover be determined by applying the image quality metric and/or the consistency metric to the 3D comparison data set.

The 3D comparison data set may advantageously be reconstructed from the plurality of training projection maps by the initial training projection matrix, e.g., by filtered backprojection.

By applying the image quality metric and/or the consistency metric to the 3D comparison data set, it is advantageously possible to provide the reference value, in particular including an image quality parameter and/or a consistency parameter. The reference value may evaluate, (e.g., qualitatively and/or quantitatively), the image quality and/or consistency of the 3D comparison data set.

By comparing the evaluation parameter with the reference value, it is advantageously possible to identify whether the 3D comparison data set has a higher image quality and/or consistency compared with the 3D-training data set. Furthermore, the at least one parameter of the trained function may advantageously be adapted such that, on repeated application of the trained function to the input data and reconstruction of the 3D training data set, the 3D training data set which may be provided in the process has a higher image quality and/or consistency compared with the 3D comparison data set.

In a further advantageous embodiment of the proposed method for providing a trained function, one initial and one further training projection matrix may be provided for each of the various projection directions. In addition, the input data of the trained function may be based on the initial training projection matrices. Furthermore, the 3D training data set may be provided through reconstruction from the training projection maps by the further training projection matrices.

Advantageously, an initial training projection matrix based on the static training model of the training X-ray device may be provided for each different projection direction of the plurality of projection directions. Advantageously, the 3D training data set may be reconstructed from the plurality of projection maps, wherein the initial training projection matrix corresponding to the projection direction of the respective projection map is used. The input data of the trained function may advantageously be based on the plurality of initial training projection matrices, in particular include the plurality of initial training projection matrices. Furthermore, the trained function may provide a further training projection matrix as output data for each different projection direction of the plurality of projection directions.

If a 3D comparison data set is being reconstructed, the 3D comparison data set may be reconstructed from the plurality of training projection maps, wherein the further training projection matrix corresponding to the projection direction of the respective projection map is used.

Using the proposed embodiment, the trained function may advantageously be configured to provide dedicated further training projection matrices for the various projection directions.

In one further advantageous embodiment of the proposed method for providing a trained function, information on dynamic degrees of freedom of movement of the training X-ray device may be acquired which defines a latent training space. The input data of the trained function may additionally be based on the latent training space.

The medical training X-ray device may have a plurality of dynamic degrees of freedom of movement. The dynamic degrees of freedom of movement may characterize an effect of friction and/or damping and/or vibration and/or moments of inertia and/or centrifugal effects and/or Coriolis effects on movement of the training X-ray device. In particular, the dynamic degrees of freedom of movement may describe a maximum influence of friction and/or damping and/or vibration and/or moments of inertia and/or centrifugal effects and/or Coriolis effects on movement of the X-ray device. The latent space may be defined, (e.g., predetermined and/or delimited), by the dynamic degrees of freedom of movement, (e.g., also by static degrees of freedom of movement), of the X-ray device. The dynamic degrees of freedom of movement, (e.g., the latent space), may describe physically possible dynamic deviations from static movements of the X-ray device. The latent space may advantageously have all the features and characteristics of the latent space described in relation to the method for providing a 3D results data set and vice versa.

The dynamic degrees of freedom of movement, (e.g., the latent training space), of the X-ray device may be learned, (e.g., by a variational autoencoder). The dynamic degrees of freedom of movement, (e.g., the latent training space_, may advantageously be ascertained based on deviations between initial training projection matrices, which are determined using the static training model of the training X-ray device, and calibration projection matrices, which are determined using calibration projection maps of a phantom of known geometry.

The input data of the trained function may advantageously additionally include the latent training space, in particular information characterizing the latent training space.

By the proposed embodiment, the trained function may advantageously be configured to provide the at least one training projection matrix while taking account of the dynamic degrees of freedom of movement of the training X-ray device.

In a further advantageous embodiment of the proposed method for providing a trained function, the training projection maps for the various projection directions may be simulated based on the static model and the latent training space.

Realistic dynamic deviations from the static model may be determined, (e.g., by measurements with a calibration phantom on the training X-ray device), resulting in the acquisition of measurement data. By the measurement data as input data, a neurological autoencoder network may be trained which describes the latent training space of the dynamic deviations. Through random modulation of input data of a decoder of the autoencoder network, any desired further realistic dynamic deviations may be generated. The measured and/or produced dynamic deviations may be combined with the static model to generate a realistic dynamic model, (e.g., the latent training space), of the training X-ray device. Realistic training projection maps may be simulated by virtual, in particular digital, forward projection of clinical 3D-image data of a training object under examination.

In a further advantageous embodiment of the proposed method for providing a trained function, the consistency metric may evaluate epipolar consistency and/or the consistency of forward projection of the 3D training data set with the training projection maps. Alternatively or additionally, the image quality metric may evaluate the total variation of the 3D comparison data set and of the 3D training data set.

The epipolar consistency condition may be based on redundancies in the captured training projection maps in the radon space, which are determined by way of geometrical parameters of the various projection directions, in particular of the 3D trajectory. If the assumed 3D trajectory does not match the training projection maps due to dynamic deviations, these deviations may be detected by way of a corresponding consistency metric. Such detection is known, for example, from the document by Robert Frysch and Georg Rose entitled, “Rigid motion compensation in interventional C-arm CT using consistency measure on projection data,” Medical Image Computing and Computer-Assisted Intervention—MICCAI 2015: 18th International Conference, Munich, Germany, Oct. 5-9, 2015, Proceedings, Part I, 18, Springer International Publishing. The consistency metric may provide an evaluation parameter for the 3D training data set, wherein a value of the evaluation parameter characterizes, in particular quantifies, the epipolar consistency of the 3D training data set.

Advantageously, virtual projection maps of the 3D training data set may in each case be determined by virtual forward projection onto the training projection maps, in particular the projection directions of the training projection maps. The consistency metric may advantageously evaluate the consistency, (e.g., correspondence), of the virtual forward projection, (e.g., of the virtual projection maps), of the 3D training data set with the corresponding training projection maps according to projection direction. The consistency metric may provide an evaluation parameter for the 3D training data set, wherein a value of the evaluation parameter characterizes, (e.g., quantifies), the consistency of the forward projection with the training projection maps.

The image quality metric may evaluate the total variation of image values, (e.g., intensity values and/or attenuation values), of the 3D training data set. The image quality metric may provide an evaluation parameter for the 3D training data set, wherein a value of the evaluation parameter characterizes, (e.g., quantifies), the total variation.

In a further advantageous embodiment of the proposed method for providing a trained function, the input data of the trained function may additionally be based on the static training model of the training X-ray device.

Advantageously, the input data of the trained function may additionally include the static training model of the training X-ray device. In this way, the trained function may advantageously be configured to provide the at least one further projection matrix while additionally taking account of the static model of the X-ray device. In particular, robust and accurate adaptation of the at least one parameter of the trained function may be provided, such that the output data of the trained function may be provided efficiently in terms of time and with high accuracy. The static training model may provide as additional input data additional weightings for determining the at least one further projection matrix, because, for example, position-dependent directional mechanical conformity values lead to main directions of dynamic effects. The weightings may be provided for individual projection directions, whereby a parameter space may be significantly reduced and robust and rapid convergence of the data-based optimization provided.

A third aspect relates to a provision unit configured to carry out a proposed method for providing a 3D results data set.

The provision unit may include at least one processor, memory, and/or an interface. The provision unit may be configured to carry out a proposed method for providing a 3D results data set in that the at least one interface, processor, and/or memory are configured to carry out the corresponding method acts.

Advantageously, the interface may be configured to acquire the projection maps and/or to provide the 3D results data set. Furthermore, the at least one processor and/or memory may be configured to provide the initial and the further training projection matrix.

The advantages of the proposed provision unit correspond to the advantages of the proposed method for providing a 3D results data set. Features, advantages, or alternative embodiments mentioned in this connection are likewise also applicable to the other claimed subjects and vice versa.

A fourth aspect relates to a medical X-ray device, including a proposed provision unit. The X-ray device is configured to capture the projection maps of the object under examination from the various projection directions, in particular along the 3D trajectory.

The advantages of the proposed X-ray device correspond to the advantages of the proposed method for providing a 3D results data set. Features, advantages, or alternative embodiments mentioned in this connection are likewise also applicable to the other claimed subjects and vice versa.

A fifth aspect relates to a training unit configured to carry out a proposed method for providing a trained function. The training unit may advantageously include at least one training interface, training memory, and/or training processor. The training unit may be configured to carry out a method for providing a trained function in that the at least one training interface, training memory, and/or training processor are configured to carry out the corresponding method acts.

Advantageously, the interface may be configured to acquire the training projection maps and/or to provide the trained function. Furthermore, the at least one processor and/or memory may be configured to provide the initial and the further training projection matrix, to reconstruct the 3D comparison data set and the 3D training data set, to determine the evaluation parameters, and to adapt the at least one parameter of the trained function.

The advantages of the proposed training unit correspond to the advantages of the proposed method for providing a trained function. Features, advantages, or alternative embodiments mentioned in this connection are likewise also applicable to the other claimed subjects and vice versa.

A sixth aspect relates to a computer program product with a computer program directly loadable into a memory of a provision unit, having program sections for carrying out all the acts of the method for providing a 3D results data set and/or to carry out one of the aspects thereof when the program sections are executed by the provision unit; and/or which is directly loadable into a training memory of a training unit, having program sections for carrying out all the acts of a proposed method for providing a trained function and/or to carry out one of the aspects thereof when the program sections are executed by the training unit.

The disclosure may furthermore relate to a computer program or computer-readable storage medium including a trained function provided by a proposed method or one of the aspects thereof.

A software-based implementation has the advantage that provision units and/or training units which are already in service may straightforwardly be retrofitted to operate in the manner according to the disclosure by a software update. In addition to the computer program, such a computer program product may optionally include additional elements such as e.g., documentation and/or additional components, as well as hardware components, such as e.g., hardware keys (dongles, etc.) for using the software.

### BRIEF DESCRIPTION OF THE DRAWINGS

Embodiments of the disclosure are shown in the drawings and described in greater detail below. Identical reference signs are used for identical features in different figures. In the figures:

FIGS. 1 and 2 are schematic representations of advantageous embodiments of a proposed method for providing a 3D results data set.

FIGS. 3 to 5 are schematic representations of advantageous embodiments of a proposed method for providing a trained function.

FIG. 6 is a schematic representation of an example of a provision unit.

FIG. 7 is a schematic representation of an example of a training unit.

FIG. 8 is a schematic representation of an example of a medical X-ray device.

### DETAILED DESCRIPTION

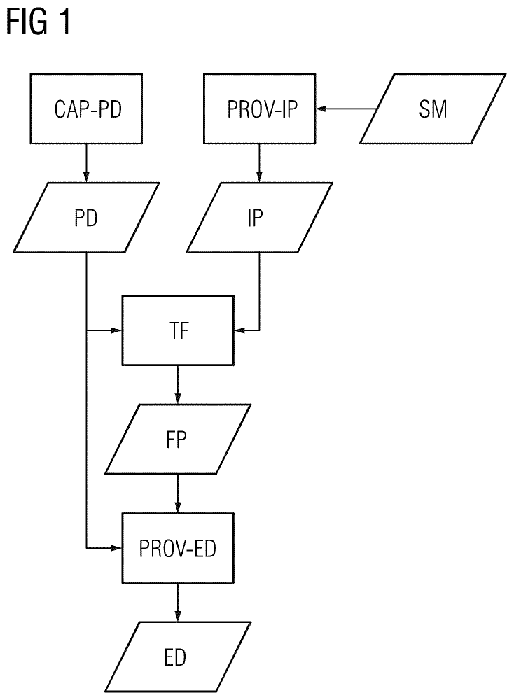

FIG. 1 is a schematic representation of an advantageous embodiment of a proposed method for providing a 3D results data set PROV-ED. In the process, projection maps PD of an object under examination may be acquired which are captured from various projection directions, in particular along a 3D trajectory, using a medical X-ray device. Furthermore, an initial projection matrix IP, in particular in each case an initial projection matrix IP for the various projection directions, may be provided PROV-IP based on a static model SM of the X-ray device. In addition, a further projection matrix FP, in particular a further projection matrix FP for each of the various projection directions, may be provided by applying a trained function TF to input data. The input data of the trained function TF may be based on the initial projection matrix IP, in particular the initial projection matrices IP, and the projection maps PD. In addition, at least one parameter of the trained function TF may be adapted based on an image quality metric and/or a consistency metric. The further projection matrix FP, in particular the further projection matrices, may be provided as output data of the trained function TF. Thereafter, the 3D results data set ED may be provided PROV-ED through reconstruction from the projection maps PD by the further projection matrix FP, in particular the further projection matrices FP.

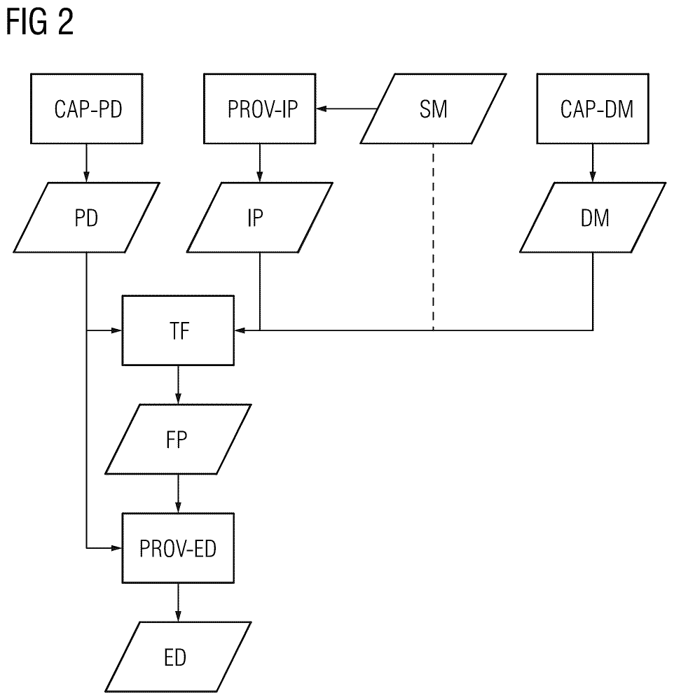

FIG. 2 shows a schematic representation of a further advantageous embodiment of the proposed method for providing a 3D results data set PROV-ED. In this case, the input data of the trained function TF may additionally be based on the static model SM of the X-ray device. Advantageously, information relating to dynamic degrees of freedom of movement of the X-ray device may be acquired CAP-DM, this defining a latent space DM. The input data of the trained function TF may additionally be based on the latent space DM.

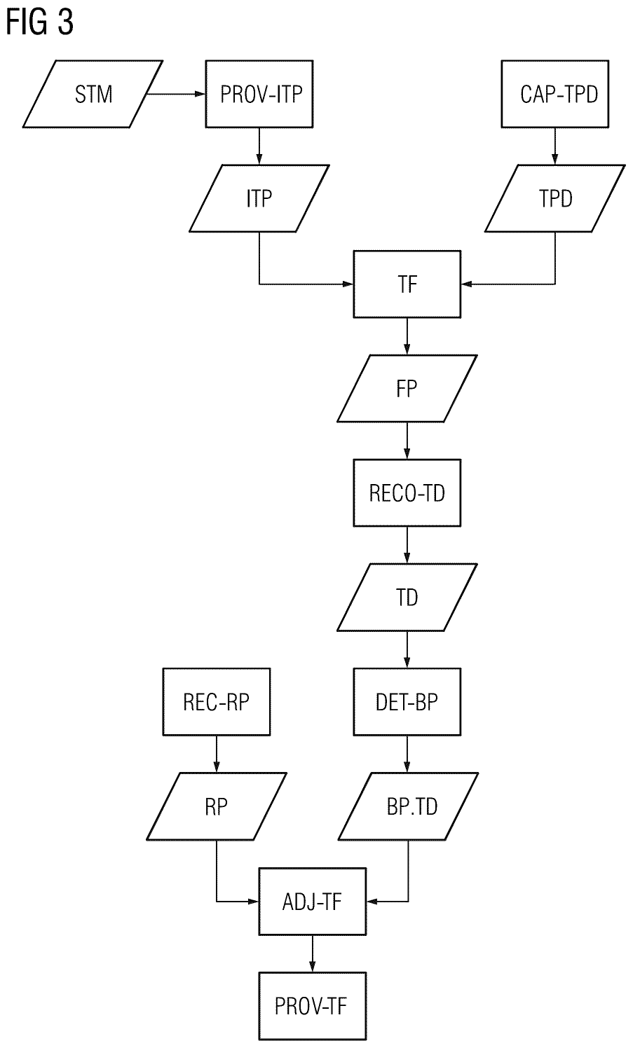

FIG. 3 is a schematic representation of an advantageous embodiment of a proposed method for providing a trained function PROV-TF. In a first act, training projection maps TPD of a training object under examination may be acquired CAP-TPD which map the training object under examination from various projection directions, in particular along a 3D trajectory. The training projection maps TPD may be captured from the various projection directions, in particular along the 3D trajectory, by the training X-ray device. Furthermore, an initial training projection matrix ITP may be provided PROV-ITP based on a static training model TM of a medical training X-ray device. In particular, an initial training projection matrix ITP may be provided for each of the various projection directions. Furthermore, a further training projection matrix IFP, in particular a further training projection matrix IFP for each of the various projection directions, may be provided by applying the trained function TF to input data. The input data of the trained function TF may be applied to the training projection maps TPD and the initial training projection matrix ITP, in particular the plurality of initial training projection matrices ITP. Furthermore, the further training projection matrix IFP, in particular the plurality of further training projection matrices IFP, may be provided as output data of the trained function TF. Advantageously, a 3D training data set TD may be reconstructed from the training projection maps TPD by the further training projection matrix IFP. Furthermore, in each case one evaluation parameter BP.VD and BP.TD may be determined DET-BP by applying an image quality metric and/or a consistency metric to the 3D training data set TD. The consistency metric may evaluate epipolar consistency and/or the consistency of the 3D training data set TD with the training projection maps TPD. Alternatively or additionally, the image quality metric may evaluate the total variation of the 3D training data set TD. Hereafter, at least one parameter of the trained function TF may be adapted ADJ-TF in such a way based on a comparison of the evaluation parameter BP.TD with a reference value RP. The reference value RP may be received, e.g., by way of a user input, and/or determined REC-RP. In addition, the trained function TF may be provided PROV-TF.

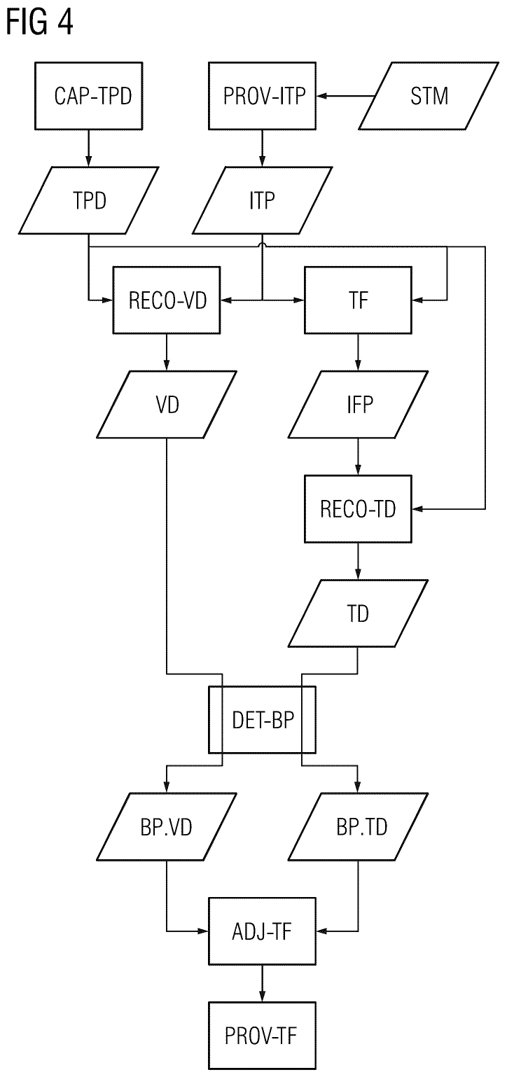

FIG. 4 is a schematic representation of a further advantageous embodiment of the proposed method for providing a trained function PROV-TF. A 3D comparison data set VD may be reconstructed RECO-VD from the training projection maps TPD by the initial training projection matrix ITP. Furthermore, the reference value RP may be determined as evaluation parameter BP.VD by applying DET-BP the image quality metric and/or the consistency metric to the 3D comparison data set VD.

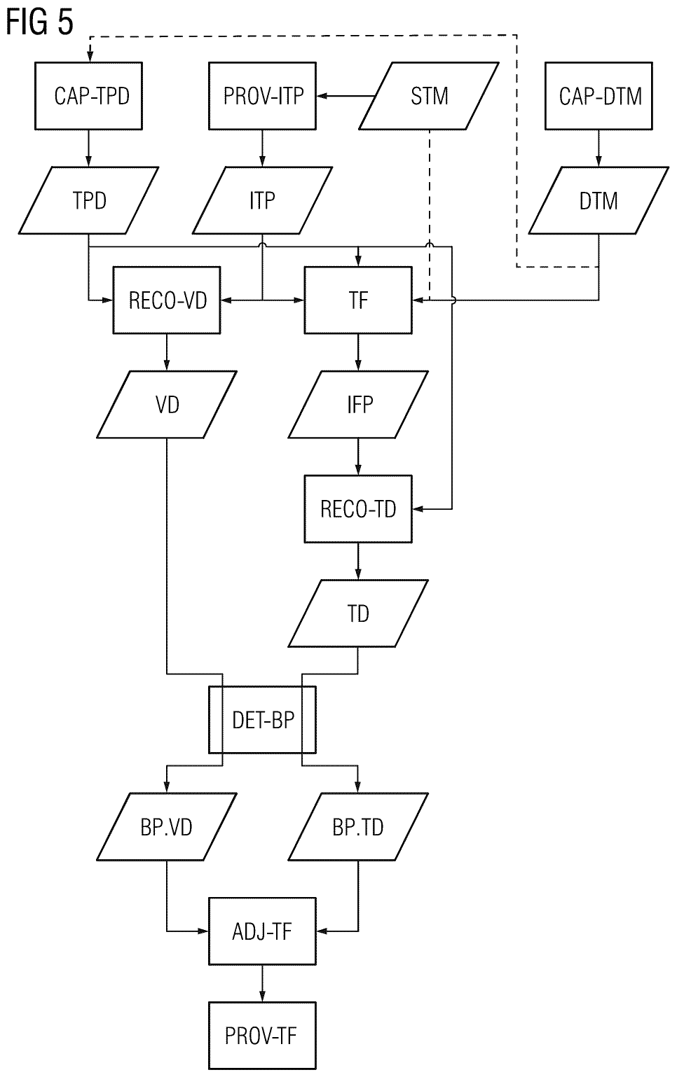

FIG. 5 is a schematic representation of a further advantageous embodiment of the proposed method for providing a trained function PROV-TF. Information relating to dynamic degrees of freedom of movement of the training X-ray device may be acquired CAP-DTM, this defining a latent training space DTM. The input data of the trained function TF may additionally be based on the latent training space DTM. In addition, the training projection maps TPD for the various projection directions may be simulated based on the static training model STM and the latent space DTM. The input data of the trained function TF may additionally be based on the static training model STM of the training X-ray device.

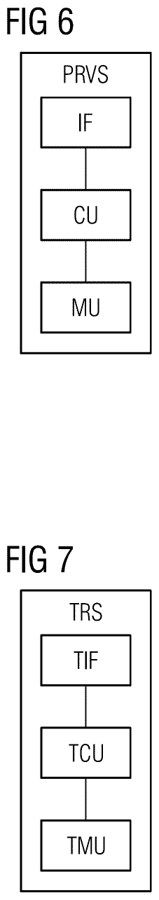

FIG. 6 is a schematic representation of a proposed provision unit PRVS. The provision unit PRVS may include a processor or computing unit CU, a memory MU, and/or an interface IF. The provision unit PRVS may be configured to carry out a proposed method for providing a 3D results data set PROV-ED, by the interface IF, the processor or computing unit CU, and/or the memory MU being configured to carry out the corresponding method acts. Advantageously, the interface IF may be configured to acquire the projection maps PD and/or to provide the 3D results data set 3D. Furthermore, the processor CU and/or the memory MU may be configured to provide the initial and the further training projection matrix IP and FP.

FIG. 7 is a schematic representation of a proposed training unit TRS. The training unit TRS may advantageously include a training interface TIF, a training memory TMU, and/or a training processor TCU. The training unit TRS may be configured to carry out a method for providing a trained function PROV-TF by the training interface TIF, the training memory TMU, and/or the training processor TCU being configured to carry out the corresponding method acts. Advantageously, the interface IF may be configured to acquire the training projection maps TPD and/or to provide PROV-TF the trained function TF. Furthermore, the processor CU and/or the memory MU may be configured to provide the initial and the further training projection matrix ITP and IFP, to reconstruct the 3D training data set TD, to determine the evaluation parameter BP.TD and to adapt ADJ-TF the at least one parameter of the trained function TF. The training interface IF may be configured to receive the reference value RP. Alternatively, the training processor TCU and/or the training memory TMU may be configured to determine the reference value RP as evaluation parameter BP.VD by applying DET-BP the image quality metric and/or consistency metric to the 3D-comparison data set VD.

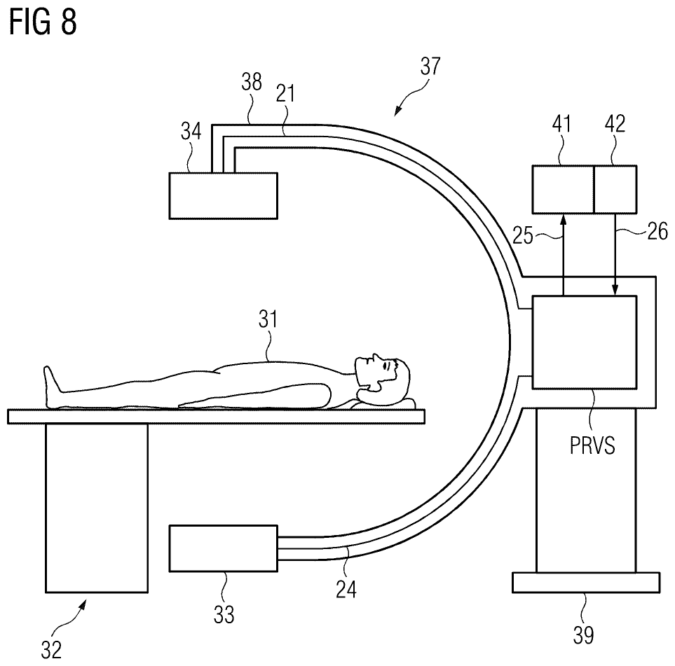

FIG. 8 shows, by way of example of a medical X-ray device, a schematic representation of a medical C-arm X-ray device 37 including a proposed provision unit PRVS. The medical C-arm X-ray device 37 may advantageously include a detector 34, in particular an X-ray detector, and a source 33, in particular an X-ray source, which are arranged in a defined arrangement on a C-arm 38. The C-arm 38 of the C-arm X-ray device 37 may be mounted in mobile manner about one or more axes. Furthermore, the C-arm X-ray device 37 may include a movement unit 39, e.g., a wheel system and/or a robot arm and/or a rail system, so enabling movement in space of the C-arm X-ray device. In order to capture the projection maps PD of the object under examination 31 positioned on a patient positioning apparatus 32, the provision unit PRVS may send a signal 24 to the X-ray source 33. Thereupon, the X-ray source 33 may emit an X-ray beam. When, after interacting with the object under examination 31, the X-ray beam impinges on a surface of the detector 34, the detector 34 may send a signal 21 to the provision unit PRVS. The provision unit PRVS may, with the assistance of the signal 21, acquire the projection maps PD.

The X-ray device may furthermore have an input unit 42, e.g., a keyboard, and a display unit 41, e.g., a monitor and/or a display and/or a projector. The input unit 42, e.g., in the case of a capacitive and/or resistive input display, may be integrated in the display unit 41. The input unit 42 may advantageously be configured for acquiring user input. To this end, the input unit 42 may e.g., send a signal 26 to the provision unit PRVS. The provision unit PRVS may be configured to control capture of the projection maps PD with the assistance of the user input. The 3D trajectory may be predetermined with the assistance of the user input.

The display unit 41 may advantageously be configured to display a graphical representation of the 3D results data set. The provision unit PRVS may to this end send a signal 25 to the display unit 41.

The schematic representations contained in the described figures do not depict any scale or size ratios.

It should again be noted that the methods described above in detail and the depicted apparatuses are merely exemplary embodiments which may be modified in the most varied manner by a person skilled in the art without departing from the scope of the disclosure. Furthermore, use of the indefinite article “a” does not rule out the possibility of a plurality of the features in question also being present. Likewise, the terms “unit” and “element” do not rule out the possibility of the components in question consisting of a plurality of interacting sub-components which may optionally also be spatially distributed.

In the context of the present application, the expression “based on” may be understood to mean “using.” In particular, wording according to which a first feature is generated (or: ascertained, determined, etc.) based on a second feature does not rule out the possibility of the first feature being generated (or: ascertained, determined, etc.) based on a third feature.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for providing a three-dimensional (3D) results data set, the method comprising:
acquiring projection maps of an object under examination that are captured from various projection directions by a medical X-ray device;
providing an initial projection matrix based on a static model of the medical X-ray device;
providing a further projection matrix by applying a trained function to input data, wherein the input data is based on the initial projection matrix and the projection maps, wherein at least one parameter of the trained function is adapted based on an image quality metric and/or a consistency metric, and wherein the further projection matrix is provided as output data of the trained function; and
providing the 3D results data set through reconstruction from the projection maps by the further projection matrix.

2. The method of claim 1, wherein an initial projection matrix and a further projection matrix are provided for each projection direction of the various projection directions,
wherein the input data of the trained function is based on the initial projection matrices, and
wherein the 3D results data set is provided through reconstruction from the projection maps by the further projection matrices.

3. The method of claim 2, wherein the input data of the trained function is additionally based on the static model of the medical X-ray device.

4. The method of claim 3, wherein information relating to dynamic degrees of freedom of movement of the medical X-ray device is acquired, therein defining a latent space, and
wherein the input data of the trained function is additionally based on the latent space.

5. The method of claim 1, wherein the input data of the trained function is additionally based on the static model of the medical X-ray device.

6. The method of claim 1, wherein information relating to dynamic degrees of freedom of movement of the medical X-ray device is acquired, therein defining a latent space, and
wherein the input data of the trained function is additionally based on the latent space.

7. A computer-implemented method for providing a trained function, the method comprising:
acquiring training projection maps of a training object under examination which map the training object under examination from various projection directions, wherein the training projection maps are simulated or captured by a medical training X-ray device;
providing an initial training projection matrix based on a static training model of the medical training X-ray device;
providing a further training projection matrix by applying the trained function to input data, wherein the input data is based on the training projection maps and the initial training projection matrix, and wherein the further training projection matrix is provided as output data of the trained function;
reconstructing a three-dimensional (3D) training data set from the training projection maps by the further training projection matrix;
determining an evaluation parameter by applying an image quality metric and/or a consistency metric to the 3D-training data set;
adapting at least one parameter of the trained function based on a comparison of the evaluation parameter with a reference value; and
providing the trained function.

8. The method of claim 7, wherein a 3D comparison data set is reconstructed from the training projection maps by the initial training projection matrix, and
wherein the reference value is determined by applying the image quality metric and/or the consistency metric to the 3D comparison data set.

9. The method of claim 8, wherein an initial training projection matrix and a further training projection matrix are provided for each projection direction of the various projection directions,
wherein the input data of the trained function is based on the initial training projection matrices, and
wherein the 3D training data set is provided through reconstruction from the training projection maps by the further training projection matrices.

10. The method of claim 9, wherein information relating to dynamic degrees of freedom of movement of the medical training X-ray device is acquired, therein defining a latent training space, and
wherein the input data of the trained function is additionally based on the latent training space.

11. The method of claim 10, wherein the training projection maps for the various projection directions are simulated based on the static training model and the latent training space.

12. The method of claim 7, wherein the consistency metric evaluates an epipolar consistency and/or a consistency of forward projections of the 3D training data set with the training projection maps, and/or
wherein the image quality metric evaluates a total variation of the 3D training data set.

13. The method of claim 7, wherein the input data of the trained function is additionally based on the static training model of the medical training X-ray device.

14. The method of claim 7, wherein information relating to dynamic degrees of freedom of movement of the medical training X-ray device is acquired, therein defining a latent training space, and
wherein the input data of the trained function is additionally based on the latent training space.

15. The method of claim 14, wherein the training projection maps for the various projection directions are simulated based on the static training model and the latent training space.

16. The method of claim 15, wherein the input data of the trained function is additionally based on the static training model of the medical training X-ray device.

17. A medical X-ray device comprising:
a provision unit configured to:
acquire projection maps of an object under examination that are captured from various projection directions by the medical X-ray device;
provide an initial projection matrix based on a static model of the medical X-ray device;
provide a further projection matrix by applying a trained function to input data, wherein the input data is based on the initial projection matrix and the projection maps, wherein at least one parameter of the trained function is adapted based on an image quality metric and/or a consistency metric, and wherein the further projection matrix is provided as output data of the trained function; and
provide a three-dimensional (3D) results data set through reconstruction from the projection maps by the further projection matrix.

18. The medical X-ray device of claim 17, wherein the medical X-ray device is configured to capture the projection maps of the object under examination from the various projection directions.

19. A training unit comprising:
at least one memory and at least one processor configured to:
acquire training projection maps of a training object under examination which map the training object under examination from various projection directions, wherein the training projection maps are simulated or captured by a medical training X-ray device;
provide an initial training projection matrix based on a static training model of the medical training X-ray device;
provide a further training projection matrix by applying a trained function to input data, wherein the input data is based on the training projection maps and the initial training projection matrix, and wherein the further training projection matrix is provided as output data of the trained function;
reconstruct a three-dimensional (3D) training data set from the training projection maps by the further training projection matrix;
determine an evaluation parameter by applying an image quality metric and/or a consistency metric to the 3D-training data set;
adapt at least one parameter of the trained function based on a comparison of the evaluation parameter with a reference value; and
provide the trained function.
